(19) United States
(12) Patent Application Publication
(10) Pub. No.: US 2007/0288020 A1
(43) Pub. Date: Dec. 13, 2007

(54) Soft tissue fixation device for organs proximate shankbone

(75) Inventors: Rong-Sen Yang, Taipei City (TW); Chao-Chang A. Chen, Taipei City (TW); Chen-Yu Lung, Taoyuan City (TW); Cheng-Kuang Lu, Keelung City (TW)

Correspondence Address: TROXELL LAW OFFICE PLLC, SUITE 1404, 5205 LEESBURG PIKE, FALLS CHURCH, VA, 22041, US

(73) Assignee: United Orthopedic Corporation

(21) Appl. No.: 11/445,317

(22) Filed: Jun. 2, 2006

(51) Int. Cl. A61F 2/30

(52) U.S. Cl. 606/69

(57) ABSTRACT

A soft tissue fixation device for organs proximate shankbone includes a fixing body, a fixing plate, and a plurality of fixing components for attaching the fixing plate to the fixing body. The fixing body forms an arcuate bound portion on a side thereof. A plurality of engaging holes is defined in a surface of the bound portion. The fixing plate is arcuate and defines at least a positioning hole substantially in a center thereof for passing a suture through, and fixing holes respectively adjacent both sides thereof for corresponding to the engaging holes. The suture and ligament are positioned reliably with respect to the fixing body. The fixing plate is adjustable in height and orientation.

### BACKGROUND OF THE INVENTION

(a) Field of the Invention

The present invention relates to a soft tissue fixation device for organs proximate shankbone, and particularly to a soft tissue fixation device for organs proximate shankbone which facilitates to position suture and ligament and to adjust orientation of a fixing plate thereof.

(b) Description of the Prior Art

A conventional soft tissue fixation device for organs proximate shankbone comprises a fixing body 5, as illustrated in FIG. 7. A half ring-shaped fixing plate 51 is provided on an appropriate position of the fixing body 5, and defines a gap 52 with respect to the fixing body 5. A surgeon ordinarily inserts a suture through ligament of a patient and then binds, or directly binds around peripheral of the ligament with the suture. Finally the suture is pulled through the gap 52 and surrounds the fixing plate 51. In this way, the ligament of the patient is fastened to the fixing plate 51.

Either the suture is pulled through the ligament and then binds, or is directly bound around peripheral of the ligament, the suture tends to slide along the fixing plate 51 since the fixing plate 51 is shaped of a half ring.

Furthermore, the fixing plate 51 is fixed on a predetermined position and cannot be adjusted in height, therefore cannot suit for diverse ligaments with different length. In addition, the suture, which is pulled through the gap 52 and surrounds the fixing plate 51, bears external force from surgeon. Once the external force is in excess, the ligament is apt to be damaged. Otherwise the suture may loosen.

A soft tissue fixation device for organs proximate shankbone is desired to overcome shortcomings mentioned above.

### SUMMARY OF THE INVENTION

Accordingly, an object of the present invention is to provide a soft tissue fixation device for organs proximate shankbone binding ligament reliably and accurately by means of combination of a fixing plate and fixing components.

Another object of the present invention is to provide a soft tissue fixation device for organs proximate shankbone which allows a fixing plate thereof adjustable appropriately according to length and orientation of ligament, thereby the ligament being adjustable, correspondingly.

A soft tissue fixation device for organs proximate shankbone of the present invention comprises a fixing body, a fixing plate, and a plurality of fixing components. The fixing body forms an arcuate bound portion on a side thereof. A plurality of engaging holes is defined in a surface of the bound portion. The fixing plate is arcuate and has radian fitting to radian of the bound portion. The fixing plate defines at least a positioning hole substantially in a center thereof for passing a suture through in a surgical operation, and fixing holes respectively adjacent both sides thereof for corresponding to the engaging holes. The fixing holes offset relative to a transverse middle line of the fixing plate. The fixing plate further defines a groove longitudinally in an inner surface thereof. The fixing components are extended into the fixing holes and the engaging holes for retaining the fixing plate onto the bound portion.

To enable a further understanding of the said objectives and the technological methods of the invention herein, the brief description of the drawings below is followed by the detailed description of the preferred embodiments.

### BRIEF DESCRIPTION OF THE DRAWINGS

FIG. 1 is an exploded view of a soft tissue fixation device for organs proximate shankbone according to the present invention.

FIG. 2 is a dimensional view of a fixing plate of the soft tissue fixation device for organs proximate shankbone of FIG. 1, wherein the fixing plate is placed in reverse orientation.

FIG. 3 is an assembled view of FIG. 1.

FIG. 4 is another assembled view of FIG. 1, wherein assembled position of the fixing plate is different from FIG. 3.

FIG. 5 shows the soft tissue fixation device for organs proximate shankbone of FIG. 1 being used in an operation.

FIG. 6 is similar to FIG. 5, wherein the operation is completed.

FIG. 7 is a dimensional view of a conventional soft tissue fixation device for organs proximate shankbone.

### DETAILED DESCRIPTION OF THE PREFERRED EMBODIMENTS

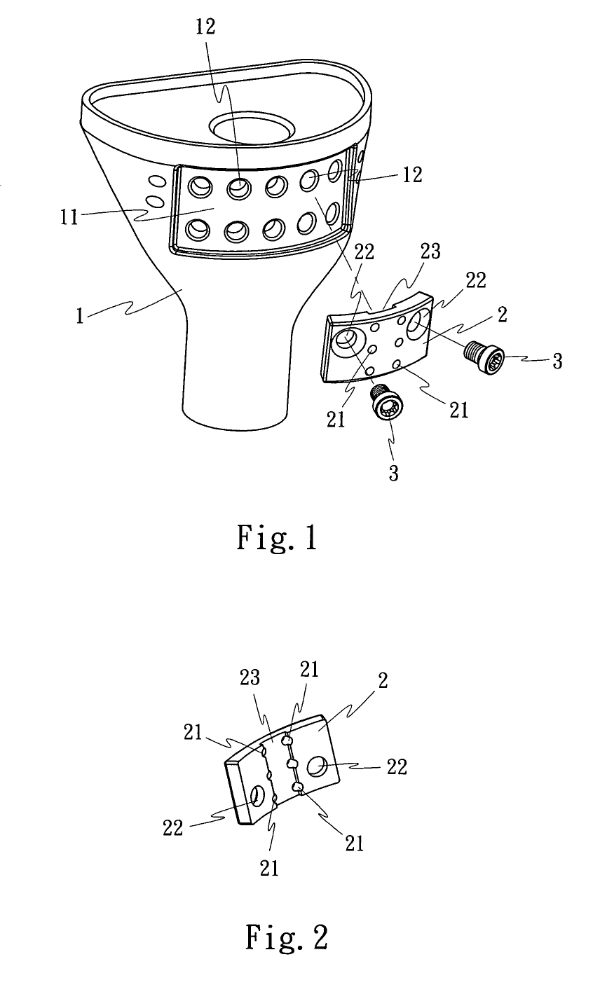

With reference to FIG. 1, a soft tissue fixation device for organs proximate shankbone according to the present invention comprises a fixing body 1, a fixing plate 2, and a plurality of fixing components 3 for attaching the fixing plate 2 to the fixing body 1.

The fixing body 1 forms an arcuate bound portion 11 on a side thereof. The bound portion 11 is slightly concave relative to an outer surface of the fixing body 1. A plurality of engaging holes 12 is defined in a surface of the bound portion 11.

The fixing plate 2 is arcuate and has radian corresponding to radian of the bound portion 11 for fitting to the bound portion 11. The fixing plate 2 defines at least a positioning hole 21 substantially in a center thereof for passing a suture through in a surgical operation, and fixing holes 22 respectively adjacent both sides thereof for corresponding to the engaging holes 12. The fixing holes 22 substantially offset relative to a transverse middle line of the fixing plate 2. Referring to FIG. 2, the fixing plate 2 further defines a groove 23 in an inner surface and through a longitudinal middle thereof.

The fixing components 3 are extended into the fixing holes 22 and the engaging holes 12, thereby retaining the fixing plate 5 onto the bound portion 11 of the fixing body 1.

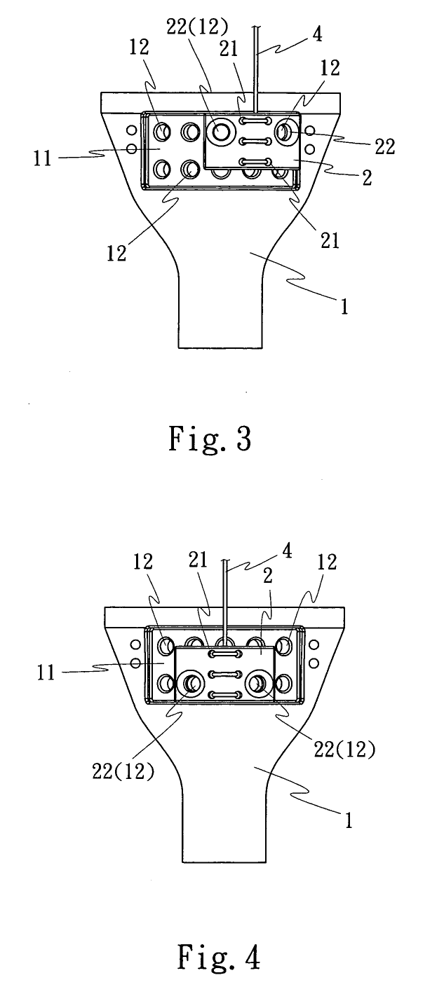

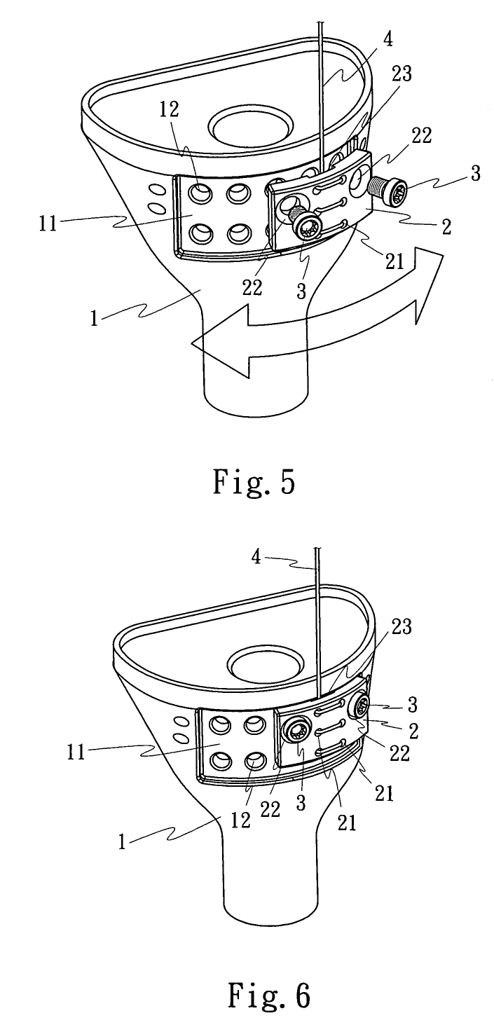

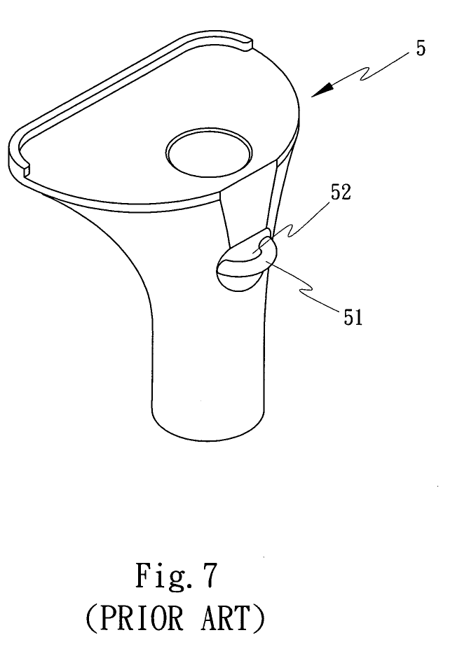

Referring to FIGS. 3 and 4, in assembly, a surgeon binds a peripheral of ligament of a patient with a suture 4. The ligament is retained on the fixing plate 2 by the suture 4. The engaging holes 12 are distributed at different height and orientation. The fixing plate 2 may be positioned in height and orientation according to length and angle of the ligament by means of selecting different engaging holes 12. The fixing holes 22 of the fixing plate 2 are respectively aligned with the selected engaging holes 12, and the fixing components 3 are extended respectively into the fixing holes 22 and the selected engaging holes 12 to attach the fixing plate 2 on the bound portion 11 of the fixing body 1, as shown in FIGS. 5 and 6. When the fixing plate 2 engages to the bound portion 11, the groove 23 of the fixing plate 2 provides space for allowing the suture 4 to pull therethrough. This facilitates the suture 4 and the ligament positioned reliably relative to the fixing body 1, and avoids damaging the ligament.

In practical use, coapting arrangement is disposed on surfaces of the fixing plate 2 to coapt the positioned ligament for facilitating growth of soft tissue. The coapting arrangement may be granulas with growth gene or hydroxyapetite coating.

The fixing holes 22 are arranged to displace relative to a transverse middle line of the fixing plate 2. Referring to FIG. 3, in the case that the fixing holes 22 are positioned relatively high and are aligned with relatively high engaging holes 12, the soft tissue fixation device for organs proximate shankbone is suitable for relatively short ligament. Otherwise, referring to FIG. 4, the fixing plate 2 turns upside down, the fixing holes 22 are positioned relatively low and are aligned with relatively low engaging holes 12, the soft tissue fixation device for organs proximate shankbone is suitable for relatively long ligament. Thus the fixing plate 2 is adjustable in height and orientation, and therefore is adapted for a variety of ligament under different conditions.

It is understood that the invention may be embodied in other forms without departing from the spirit thereof. Thus, the present examples and embodiments are to be considered in all respects as illustrative and not restrictive, and the invention is not to be limited to the details given herein.

What is claimed is:

1. A soft tissue fixation device for organs proximate shankbone comprising:
a fixing body forming an arcuate bound portion on a side thereof, a plurality of engaging holes being defined in a surface of the bound portion;
a fixing plate being arcuate with radian corresponding to radian of the bound portion for fitting to the bound portion, and defining at least a positioning hole substantially in a center thereof for passing a suture through, and fixing holes respectively adjacent both sides thereof for corresponding to the engaging holes, a groove being longitudinally defined in an inner surface of the fixing plate; and
a plurality of fixing components being extended into the fixing holes and the engaging holes for retaining the fixing plate onto the bound portion.

2. The soft tissue fixation device for organs proximate shankbone as claimed in claim 1, wherein the bound portion is slightly concave relative to an outer surface of the fixing body.

3. The soft tissue fixation device for organs proximate shankbone as claimed in claim 1, wherein coapting arrangement is disposed on surfaces of the fixing plate to coapt positioned ligament for facilitating growth of soft tissue.

4. The soft tissue fixation device for organs proximate shankbone as claimed in claim 3, wherein the coapting arrangement is hydroxyapetite coating.

5. The soft tissue fixation device for organs proximate shankbone as claimed in claim 1, wherein the fixing holes offset relative to a transverse middle line of the fixing plate.
